(19) United States
(12) Patent Application Publication
(10) Pub. No.: US 2002/0029032 A1
(43) Pub. Date: Mar. 7, 2002

(54) Fluorescent surgical hardware and surgical supplies for improved visualization

(76) Inventor: Eva Arkin, Atlanta, GA (US)

Correspondence Address: John K. McDonald, Ph.D., KILPATRICK STOCKTON LLP, Suite 2800, 1100 Peachtree Street, Atlanta, GA, 30309-4530, US

(21) Appl. No.: 09/948,352

(22) Filed: Sep. 7, 2001

Related U.S. Application Data
(60) Provisional application No. 60/231,176, filed on Sep. 7, 2000.

(51) Int. Cl. A61B 17/00

(52) U.S. Cl. 606/1

(57) ABSTRACT

This invention provides surgical articles, including needles, other hardware, and surgical supplies that include an element that fluoresces upon exposure to ultraviolet light. This quality makes the surgical articles more visible against their background, and facilitates the location of missing items that may be inadvertently dropped or retained during surgery, and also facilitates the inspection of items placed in surgery.

### CROSS REFERENCE TO RELATED APPLICATION

This patent application claims priority to U.S. Provisional Patent Application Ser. No. 60/231,176, filed Sep. 7, 2000.

### FIELD OF THE INVENTION

This invention provides surgical articles such as needles, other hardware, and surgical supplies that include an element that fluoresces upon exposure to ultraviolet light. This quality makes the items more visible against their background, and facilitates the location of missing items that may be inadvertently dropped or retained during surgery.

### BACKGROUND OF THE INVENTION

Contemporary surgical procedures involve the use of large numbers of instruments and supplies. Despite elaborate procedures to account for these items at the conclusion of each surgical procedure, a significant problem remains involving surgical articles that are inadvertently lost during surgery and possibly retained within the patient. Retained foreign objects from surgery can be a source of considerable post-operative pain, may injure other anatomic structures, and may cause infection or foreign body reactions. Because the risks of injury and attendant liability are so great, most hospitals have elaborate protocols for unaccounted-for items that require an exhaustive search of the operative field and entire surgical theater before closure of the incisions.

As a general hospital protocol in the United States, an absolute count of the entire contents of the instrument supply table is maintained for each surgical procedure. This accounting covers all items that are available for use by the surgeon, including all surgical instruments, needles, blades, other hardware, containers, sutures, medications, syringes, surgical implants, and all absorbent items (such as gauze pads, cotton balls, and towels). The whereabouts of each article that enters the sterile surgical field must be accounted for both during and after surgeries. As each surgical layer is closed, for example, the surgical team must account for the whereabouts of each article used to that point. A single missing and unaccounted for sponge or needle means costly delays while a search for the missing item is conducted. X-ray machines may have to be brought into the operating room to x-ray the patient in order to determine if a missing sponge or needle was inadvertently closed within the patient. Identification of a retained item on x-ray generally requires that any closed layers be reopened until the missing article is physically located.

In a typical operating room situation, the nursing/technical personnel manually count and recount each article that is placed on the sterile supply table before every operation. Any additional instruments and supplies that are obtained during the procedure are added to the tally as they are placed on the sterile supply table. As the surgeon begins to close the deeper aspects of the surgical procedure, a recounting is begun, so that all articles can be accounted for before the surgical incisions are finally sealed. If any items are missing on the final count, the count is repeated. If an article remains missing, a thorough visual search is conducted. The surgeon is informed and searches within the operative site, while the nursing and technical personnel search the supply and instrument tables, the drapes covering the operating table, and the floor surrounding the operating and supply tables. If the article is not located, then the surgeon is typically expected to obtain intra-operative x-rays of the patient to ascertain that the item is not internally retained. In most institutions, the surgeon can avoid the use of x-rays only by assuming responsibility for stating that the article in question physically could not be within the surgical site (six inch scissors in an eye surgical wound, for example). Unaccounted-for surgical articles then require completion of written incident reports that generally must be signed by the surgeon and the operating room personnel involved. These reports are then reviewed by internal hospital oversight committees, thereby increasing costs of valuable professional and clerical time that is attributed to the lost surgical article.

Despite elaborate preventative protocols, the problem of lost surgical articles remains a major issue even at some of the most highly regarded clinical facilities. A published survey by the Risk Management Foundation for the Harvard Medical Institutions for all malpractice claims filed against Harvard-affiliated institutions from 1976 to 1993 revealed that the third most common allegation was “retained foreign body.”

Two to three hundred, standard-sized 4×4 inch surgical gauze sponges may be used in a typical surgery. Approximately four million packages of ten-count, 4×4 inch surgical gauze sponges are used in surgeries in the United States each year. The surgical team must individually account for each one of these sponges that enters a sterile surgical field before a surgery is completed.

Beyond the liability concerns, there is a significant economic cost related to the issue of missing surgical articles. Intra-operative radiographs must be obtained to demonstrate that the missing item is not within the operative body space. The additional search and x-ray time can add to health costs, as operating room and anesthesia charges are typically billed in quarter hour increments. In addition, the patient is exposed to the effects of the additional anesthesia, beyond that required for the surgery. Multiple studies have correlated increased detrimental risk with increased exposure to anesthesia. As discussed above, missing surgical items also impose an expensive burden on the hospital's risk management procedures, requiring preparation and review of incident reports by valuable nursing and surgical personnel. Finally, when a missing surgical article cannot be located, a liability risk may yet remain for an article that may be radiolucent or too small to visualize on x-rays.

An additional aspect of the economic cost of lost surgical items relates to surgical prostheses that are used in various types of microsurgery. As an example, ear surgery may involve use of ossicular implants crafted of various metals, ceramics, or plastics that serve to replace some or all of the bones of the middle ear that conduct sound from the eardrum to the inner ear. These implants may involve elaborate fabrication, and are often quite small (lengths of 4 mm or less are common). When these implants are lost during handling by surgical personnel, replacement implants must be used. If the lost implants could be located, in some circumstances they could be returned to their manufacturers for re-sterilization. With implants that may cost hundreds of dollars or more, an improved recovery mechanism for lost articles would result in economic savings.

Prior art efforts at improved needle visualization have been limited to the direct visualization of the items, generally by coloring the items. Because these items are often quite small, they may still be lost against a complex background of surface textures and colors. Surgical sponges have contained woven filaments of radio-opaque material for years, but even these tracers can occasionally result in a mistaken radiologic diagnosis. Therefore, the need exists for an improved mechanism of locating surgical instruments and supplies.

### SUMMARY OF THE INVENTION

The present invention solves the problem described above by providing improved means and methods of visually locating items that are used and possibly lost in the course of surgery, thus reducing the potential risk of these articles being retained within the patient. The present invention relates generally to a component that may be incorporated into a variety of surgical articles to facilitate the localization of such missing articles with reduced time, expense, and risks. In particular, the invention relates to a variety of surgical articles that incorporate elements capable of fluorescence when illuminated by an ultraviolet light source.

More specifically, the present invention provides a coating for metallic articles that does not interfere with the mechanical function of the articles, and is capable of fluorescence when illuminated by an ultraviolet light source.

Similarly, for non-metallic articles, the present invention provides for either a coating or the structural inclusion of compounds that may fluoresce when illuminated by an ultraviolet light source.

Thus, it is an object of the present invention to facilitate the identification of small items of surgical hardware and supplies that may become lost during surgical procedures, thereby preventing their inadvertent and possibly detrimental retention within the body.

It is a further object of the present invention to reduce the economic and other potential costs associated with prolonged searches for missing surgical items during operative procedures.

It is another object of the present invention to reduce the risk of patient injury from unintended, retained surgical articles.

Still another object of the present invention is to reduce the liability risks for health care providers associated with surgical articles lost within a patient during surgery.

It is yet another object of the present invention to facilitate the identification and removal of implantable drug delivery devices during surgery.

It is yet another object of the present invention to provide an improved method whereby a surgeon can visually inspect the placement and position of surgical articles such as sutures, fasteners, retainers, and prostheses before final closure of the surgical wound.

Other objects, features, and advantages of the present invention will become apparent upon reading the following detailed description of the preferred embodiments of the invention when taken in conjunction with the drawings and appended claims.

### BRIEF DESCRIPTION OF THE DRAWINGS

Referring to the drawings:

FIG. 1 is an illustration of an exemplary embodiment of the present invention in which a fluorescent-coated surgical needle is illuminated by a conventional ultraviolet light source.

FIG. 2 is an illustration of an exemplary embodiment of the present invention in which a fluorescent-coated surgical screw is illuminated by a conventional ultraviolet light source.

FIG. 3 is an illustration of an exemplary embodiment of the present invention in which a surgical gauze pad containing a fluorescent component is illuminated by a conventional ultraviolet light source.

FIG. 4 is an illustration of an exemplary embodiment of the present invention in which a fluorescent-coated cupped piston ossicular implant is illuminated by a conventional ultraviolet light source.

FIG. 5 is an illustration of an exemplary embodiment of the present invention in which a fluorescent-coated drug delivery implant is illuminated by a conventional ultraviolet light source during surgical removal of the implant device.

### DETAILED DESCRIPTION OF THE PREFERRED EMBODIMENTS

The present invention is directed to applications involving surgical articles. Surgical articles include any item used in surgery. Surgical articles include, but are not limited to, instruments, implants, hardware, devices, and absorbent materials. In the context of the present invention as disclosed herewith, such a surgical article may be a surgical needle, screw, wire, pin, clip, staple, spring, or rivet. In other embodiments of the present invention, such a surgical article may be a surgical instrument or implement, or component part thereof. In still other embodiments according to the present invention, such a surgical article may be a surgical implant, prosthesis, or an implantable drug delivery device. In yet other embodiments of the present invention, the term “surgical article” may refer to a surgical sponge, pad, mesh, stent, catheter, or suture. Within the various embodiments contemplated by the present invention, the surgical article may contain elements constructed of metallic, ceramic, plastic, fibrous materials, or combinations thereof. Finally, within the context of the present invention as disclosed herein, elements comprising the surgical article may be solid, woven, nonwoven, braided, or monofilamental in their structure.

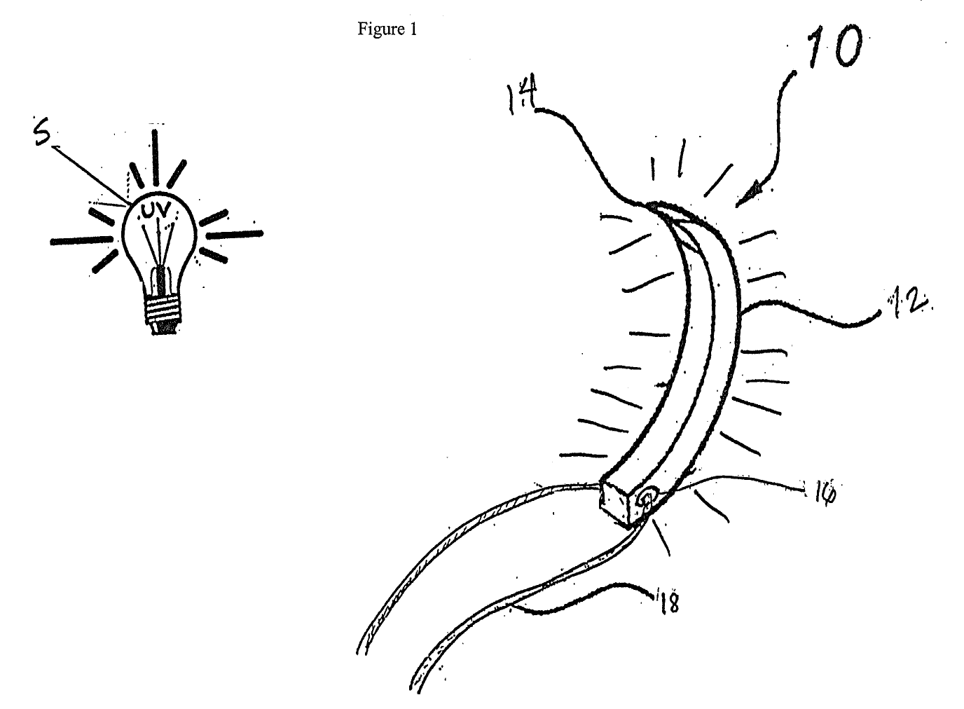

An exemplary embodiment of the present invention is shown in FIG. 1. A curved surgical needle 10 comprises a body portion 12 which may taper towards a sharpened point 14 which may be finished with either a cutting or a rounded surface. At the end of the body portion 12 opposite the point 14, an eye 16 may be provided through the thickness of the body portion 12, said eye 16 sized to receive a strand of suture material 18 which may freely pass therethrough. The needle body portion 10 through some or all of its length may have a finish coating which is capable of fluorescence when it is illuminated by a standard ultraviolet light source 5. The needle 10 may be preferably fabricated of steel, but other metals or synthetic substances may be used for specific surgical applications.

Alternately, the eye 16 may be replaced by a lumen or longitudinal channel within the body portion 10 in which the suture material 18 may be received and permanently affixed to the needle body portion 10 by a compressed friction fit. Also, in alternative embodiments of the present invention, the body portion 12 of the needle 10 may be straight or curved through some or all of its length. Furthermore, in alternate embodiments of the present invention, the body portion 12 of the needle 10 may be rounded or flattened through some or all of its length.

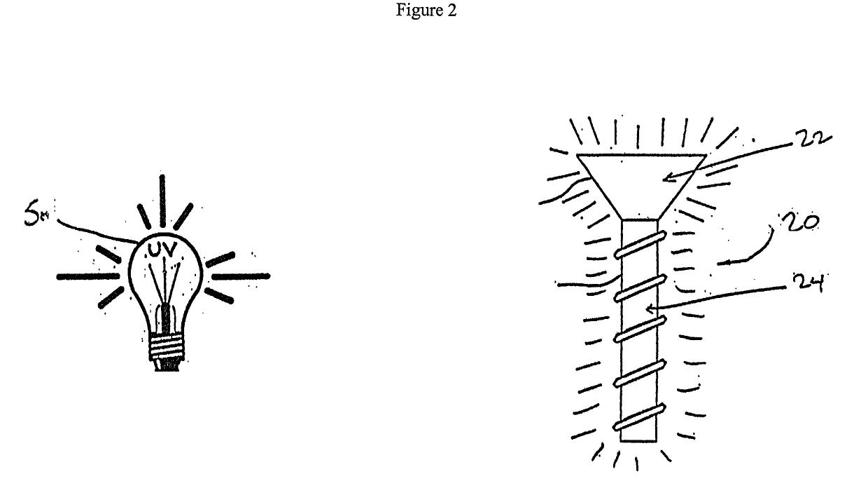

In another preferred embodiment of the present invention, as shown in FIG. 2, a metal surgical screw 20 comprises a head portion 22 which may contain a slot or other driving means and a threaded body portion 24. Said surgical screw 20, through some or all of its length, may have a finish coating which fluoresces when illuminated by a standard ultraviolet light source 5. The surgical screw 20 may serve for internal fixation applications, or may be a means to secure some other surgical appliance. Alternately, the surgical screw 20 may be a component in a mechanical surgical instrument that could become separated from the instrument and lost during surgery. In alternate embodiments of the present invention, the surgical screw 20 may comprise a setscrew in which a driving means may be located within the diameter of the threaded body portion 24, obviating the need for a head portion.

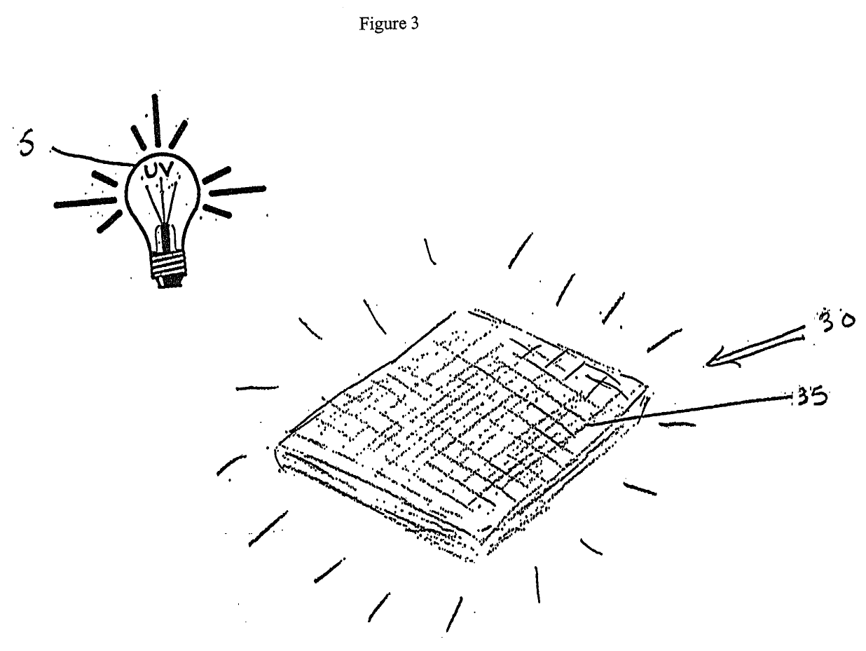

FIG. 3 illustrates another embodiment of the present invention, in which a square of surgical gauze 30 contains one or more filaments 35 of a material capable of fluorescence when illuminated by a standard ultraviolet light source 5. Alternately, one or more filaments 35 might be coated with a material capable of fluorescence. In still other alternate embodiments of the present invention, fluorescent materials might be structurally integrated with the filaments 35 interwoven in surgical gauze 30. In yet other embodiments of the present invention, conventional surgical gauze 30 might be coated with a fluorescent material to provide for visualization with ultraviolet light. Alternate embodiments of this invention also include other absorbent wicks and sponges used in surgery, in which a similar polymeric material may be structurally included or coated to provide for fluorescent visualization. In addition, coating or incorporation of a similar fluorescent polymeric material into suture material facilitates visual inspection by the surgeon to ascertain optimal suture placement before the surgery is concluded.

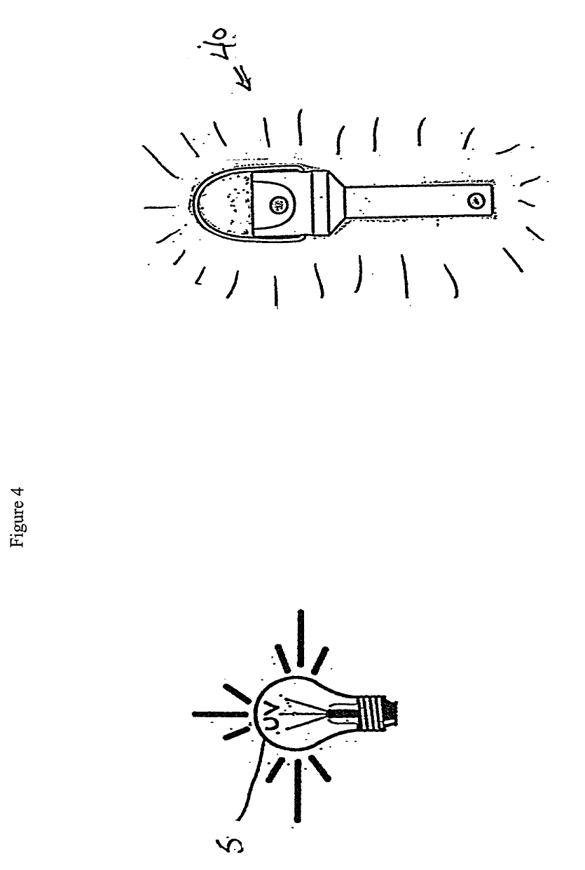

Yet another exemplary embodiment of the present invention is shown in FIG. 4, in which a metal cupped piston ossicular prosthesis 40 for middle ear surgery may be provided with a surface coating which fluoresces when illuminated by a standard ultraviolet light source 5. In this embodiment, the fluorescent quality may be used not only to locate a lost prosthesis in surgery, but also to facilitate final inspection by the surgeon to ascertain optimal positioning before the surgery is concluded.

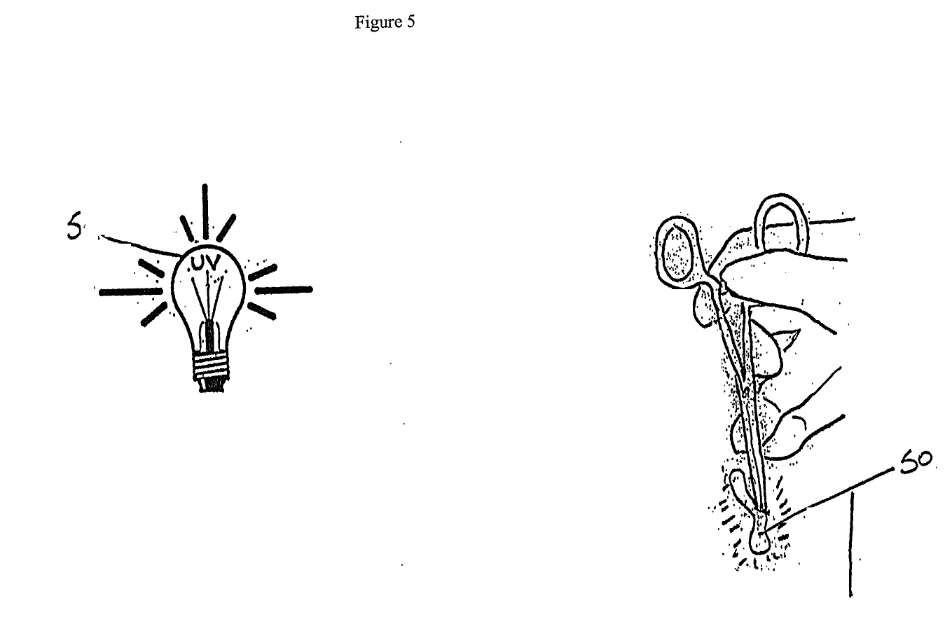

FIG. 5 shows another embodiment of the present invention, in which an implantable drug delivery device 50 may be provided with a surface coating capable of fluorescence when illuminated by a standard ultraviolet light source 5, thus facilitating the often difficult process of locating the implant for surgical removal.

Other embodiments of the present invention include other items of small hardware either used in surgery or elements of surgical instruments that could be lost during operative procedures. Such non-limiting items include, but are not limited to, blades, wires, pins, clips, staples, springs, and rivets. In addition, incorporation of a similarly fluorescent material into surgical fasteners and fixation devices facilitates visual inspection by the surgeon to ascertain their optimal positioning before the surgery is concluded.

Alternative embodiments of this invention include, but are not limited to, other implants, such as implantable pump components, cardiovascular stents, electronic leads, catheters, and other surgically implanted articles, in which a fluorescent polymeric or other fluorescent material may be incorporated to provide for fluorescent visualization to facilitate their subsequent surgical location and/or removal.

The fluorescent coatings utilized in the exemplary embodiments of the present invention may be applied to metal, ceramic, or plastic items utilizing a variety of processes, including plasma or flame spray coating methods described in U.S. Pat. No. 4,327,120. Other coating techniques known to one skilled in the art may be employed, including, but not limited to, spray, immersion, paint, roll coating, or flow coating techniques.

In alternative embodiments according to the present invention, fluorescent material may be incorporated into the structure of surgical articles, rather than applied as a coating. Such surgical articles with incorporated fluorescent materials may be constructed of any material, including but not limited to, ceramics, plastics, polymers, gels, fibers, metallic powders, or other powders. Such fluorescent materials may be bonded to, or simply dispersed within, said structural materials.

Flame sprayable or plasma sprayable metal powders can also be utilized in the practice of the present invention. These metal powders can have a particle size of from about 1 to 50 microns, preferably about 10 to 25 microns and include, for example, Co, Cr, Mo, Ni, and W. Alloy powders can also be used. The 450 Ni powder used in U.S. Pat. No. 4,327,120 is a non-limiting example of one such alloy. These alloys include Ni-Cr alloys, Fe-Cr-Ni stainless alloys and Co-based alloys. The alloy powders are generally in the same size range as the metal powders.

The phosphors which can be employed in combination with metal oxides, metal carbides, or metal powders including such materials as yttrium oxide doped with ⁺³europium, Ce_{l-x-y}La_{y}Tb_{y}MgAl₁₁O₁₉, where 0<X<9.2 and 0.2<Y<0.4, and specifically Ce_{.7}Tb_{.3}MgAl₁₁O₁₉, (CAT). Additional useful phosphors are, for example, Zn₂SiO₄, doped with Mn or As, La₂O₂S doped with Th, YVO₄ doped with Eu, Y₂O₃ doped with Eu, Y₂O₂S doped with Eu, CaWO₄, ZnS doped with Ag or Cu, ZnCdS doped with Cu or Ag, KMgF₃ doped with Mn, Gd₂O₂S doped with Th. About 1 to 75% by volume of metal oxide phosphor can be used, based on the total volume of the mixture of metal oxide phosphor and hardcoat material that may be applied to the substrate.

Fluorescence can be determined by measuring the difference between the light emitted from a surface substantially free of phosphor with a surface having an effective amount of phosphor, as disclosed in U.S. Pat. No. 4,327,120. A standard 256 nm UV lamp, held at a distance sufficient to provide a light intensity of about 1200 μwatts per cm² will show an increase of at least 0.1 ft. Lamberts over background when used on a surface derived from a sprayable mixture containing an effective amount of UV sensitive phosphors. Compared to a coating derived from a mixture free of phosphor, intensities of over 80 ft. Lamberts have been recorded from surfaces sprayed with UV sensitive phosphors.

In the practice of the present invention, a metal substrate can be plasma or flame-sprayed to a thickness of 100 microns or more with WV sensitive indicating mixture, which hereinafter will signify a mixture of the above described metal oxide phosphor with a hardcoat material, such as metal powder, metal oxide powder, or metal carbide powder as previously defined. Plasma temperatures and the corresponding particle residence time must be sufficient such that melting of each species occurs. A detailed description of the conditions used in conventional plasma or flame spraying can be found in U.S. Pat. No. 4,055,705 and 4,095,003.

Particulated metal oxides and UV sensitive metal oxide phosphors which can be plasma or flame-sprayed onto the surface of metallic substrates in accordance with the method of the present invention can have an average particle size of from about 5 to 150 microns and preferably from about 25 to 100 microns. Compounds such as Al₁₂O₃, BaTiO₃, CeO₂, Cr₂O₃, MgO, TiO₂, ZrO₂, and ZrSiO₂ are included among the powdered metal oxides.

Metal carbide phosphors may also be utilized as fluorescent compounds in the practice of the present invention. These metal carbide powders have a particle size of from about 2 to 150 microns and preferably from about 5 to 110 microns. Such metal carbides include, for example, CrC, HfC, ZrC, and WC.

Various procedures can be used to adjust the size of the metal oxide phosphor particles utilized in the present invention. One method is by spray drying as shown in U.S. Pat. Nos. 3,373,119 and 3,429,962. The spray drying process involves suspending the metal oxide phosphor in a solvent to form a slip and then spray drying the resulting mixture into a hot drum. The fluid of the slip evaporates leaving powder particles having a broad particle size range. The spray-dried particles can then be screened to eliminate the particles that do not fall within a desired size range. If desired, such spray drying procedures are used to adjust the particle size of other metal oxides free of a UV sensitive phosphor.

In addition to the compounds mentioned above, additional fluorescent compounds utilized as coatings or elements in the various embodiments of the present invention include, but are not limited to, all compounds listed in the Handbook of Fluorescent Probes and Research Chemicals, by Richard P. Haugland, Sixth Edition, Molecular Probes, Inc., Eugene, Oreg.

The above description is intended to be illustrative and not restrictive. Many embodiments will be apparent to those of skill in the art upon reading the above description. The scope of the invention should therefore be determined not with reference to the above description, but should instead be determined with reference to the appended claims, along with the full scope of equivalents to which such claims are entitled. The disclosures of all articles and references referred to herein, including patents, patent applications, and publications, are incorporated herein by reference in their entirety.

### EXAMPLE 1

As an exemplary application of the present invention, an appendectomy is a commonly performed surgical procedure that generally requires about thirty minutes of time for the surgical procedure, from incision to wound closure. Pre- and post-operative preparations generally require an additional half-hour, making a total of one hour of operating room time for which the patient would normally be charged.

If a surgical needle is missing on the final nursing count under traditional circumstances, then both the surgeon and the nursing personnel independently search the surgical field, supply tables and surrounding floor. If the needle can not be located, a portable x-ray is obtained, and the patient is kept under general anesthesia on the operating table until the x-ray is taken, processed, and interpreted. If no evidence of the needle is seen on the abdominal x-ray, then it is presumed that the needle was lost outside the patient, and the patient is awakened and transferred from the operating room. If the needle is evident on the x-ray, then the incision is re-opened and directly explored by the surgeon. The additional search time is about 15 or 20 minutes, with an x-ray adding at least another thirty minutes, for a total of 45-50 minutes of additional anesthesia and time in the operating room. Therefore, the anesthesia exposure time doubles, as does the time-based charges for anesthesia care and operating room time.

By performing an appendectomy with the fluorescent-coated needles of the present invention, a missing needle is located rapidly by dimming the overhead room lights, and using a UV lamp to examine the operating table and its environs. UV-fluorescence promptly identifies the coated needle, even on a variegated flooring surface. By using the fluorescent-coated needle, the total additional time expenditure resulting from the dropped needle is reduced to approximately 10 minutes, resulting in considerable savings and decreased exposure to anesthesia.

### EXAMPLE 2

An additional example of an application of the present invention is the facilitation of inspection of suture placements by the surgeon using fluorescent suture. Intestinal surgery generally involves a three-layered closure when it is necessary to re-anastamose two cut ends of the intestines. The sutures are typically placed in a circumferential manner, and must be evenly placed through certain layers for optimal closure.

By dimming the overhead room lights and using a UV lamp to examine the suture lines, the surgeon evaluates each layer of closure before proceeding to the next. UV-fluorescence promptly identifies improperly placed suture, allowing the surgeon to replace or reinforce any areas of sub-optimal closure.

All patents, publications and abstracts cited above are incorporated herein by referenced in their entirety. It should be understood that the foregoing relates only to preferred embodiments of the present invention and that numerous modifications or alterations may be made therein without departing from the spirit and the scope of the present invention as defined in the following claims.

What is claimed is:

1. An article for use during surgery, the article comprising:
one or more elements, wherein at least one element is capable of fluorescence when illuminated by a light source to facilitate visualization of the element during surgery.

2. The article of claim 1, wherein the at least one element capable of fluorescence comprises a fluorescent coating applied thereto.

3. The article of claim 1, wherein the at least one element capable of fluorescence comprises a fluorescent material incorporated within the element.

4. The article of claim 1, wherein the light source is a source of ultraviolet radiation.

5. The article of claim 1, wherein the article is a surgical instrument, surgical implant, item of surgical hardware, surgical device, or surgical absorbant material.

6. The article of claim 1, wherein the article is a surgical needle, screw, wire, pin, clip, staple, spring, or rivet.

7. The article of claim 1, wherein the article is a surgical sponge, pad, mesh, stent, catheter, or suture.

8. The article of claim 1, wherein the article is a surgical instrument or component thereof.

9. The article of claim 11, wherein the article is an implantable drug delivery device.

10. The element of claim 1, wherein the at least one element capable of fluorescence is solid.

11. The element of claim 1, wherein the at least one element capable of fluorescence is woven.

12. The element of claim 1, wherein the at least one element capable of fluorescence is nonwoven.

13. The element of claim 11, wherein the at least one element capable of fluorescence is braided.

14. The element of claim 1, wherein the at least one element capable of fluorescence is monofilamental.

15. A method of visualizing an article by a surgeon or other surgical personnel during surgery upon a human or animal body in a surgical environment, comprising:
(a) using an article containing one or more elements capable of fluorescence;
(b) illuminating the surgical environment with a light source; and
(c) the user visually identifying the article containing the one or more fluorescing elements of the article.

16. The method of claim 15, wherein the surgeon or other surgical personnel visualizes the article containing the fluorescing element thereby preventing inadvertent inclusion and retention of the article within the body following the surgery.

17. A method comprising:
(a) a surgeon or other surgical personnel placing and orienting an article containing one or more elements capable of fluorescence as an implant within a body during surgery upon the body;
(b) illuminating with a light source;
(c) visualizing the article containing the fluorescing element to locate the article; and,
(d) ascertaining a desired placement and orientation of the article.

18. The method of claim 15, wherein the light source is a source of ultraviolet radiation.
